(19) United States
(12) Patent Application Publication
(10) Pub. No.: US 2022/0112267 A1
(43) Pub. Date: Apr. 14, 2022

(54) PRODUCE AND ISOLATE AND/OR EXTRACT COLLAGEN AND/OR GELATIN FROM ANIMAL CELL LINES AND/OR TISSUE EXPLANTS

(71) Applicant: Jellatech, Inc., Raleigh, NC (US)

(72) Inventors: Stephanie Michelsen, Redwood City, CA (US); Kylie van Deinsen-Hesp, Wageningen (NL); Ryan Bethencourt, Durham, NC (US); Andrew Ive, Princeton, NJ (US)

(73) Assignee: Jellatech, Inc., Raleigh, NC (US)

(21) Appl. No.: 17/497,455

(22) Filed: Oct. 8, 2021

Related U.S. Application Data
(60) Provisional application No. 63/089,285, filed on Oct. 8, 2020.

(51) Int. Cl. C07K 14/78; G01N 33/50; G01N 21/31

(52) U.S. Cl. CPC C07K 14/78; G01N 21/31; G01N 33/5038

(57) ABSTRACT

Methods to establish and/or culture a (continuous) cell line of an animal, methods to engineer and/or genetically modify the (continuous) cell line of the animal, methods to isolate collagen from the (continuous) cell line of the animal, methods to modify and/or genetically engineer DNA sequences of collagen for applications and/or to increase the production of the collagen in cells of the animal, and methods to extract collagen from a cultured animal explant through use of a material and/or a process are described herein. In preferred examples, the animal is a jellyfish and the animal explant is a jellyfish explant, a jellyfish polyp explant, a jellyfish medusae explant, or a marine sponge explant.

### CROSS-REFERENCE TO RELATED APPLICATIONS SECTION

This application is a U.S. Non-Provisional patent application that claims priority to U.S. Provisional Patent Application Ser. 63/089,285 filed on Oct. 8, 2020, the entire contents of which are hereby incorporated by reference in their entirety.

### FIELD OF THE EMBODIMENTS

The field of the invention and its embodiments relate to methods to produce collagen and/or gelatin in animal cell lines and/or tissue explants and isolate and/or extract collagen and/or gelatin from said animal cell lines and/or tissue explants. In particular, the present invention relates to methods produce collagen and/or gelatin in marine animal cell lines and/or tissue explants and isolate and/or extract collagen and/or gelatin from said marine animal cell lines and/or tissue explants.

### BACKGROUND OF THE EMBODIMENTS

Collagen is one of the most abundant components of animal tissues. Collagen products find numerous medicinal and bioengineering uses. For example, collagen is used for wound dressings, as matrices for tissue growth, and as biomaterials for cosmetic surgery, reconstructive surgery, drug delivery system, and scientific researches. Most of the collagen products used in these fields are derived from bovine or porcine tissue. Additionally, many procedures describing extraction of collagen from vertebrates, such as cattle, pigs, horses, sheep, poultry, whales, sharks, fish are known. However, such methods harm the animal. As such, improved humane methods to produce collagen and/or gelatin in animal cell lines and/or tissue explants and isolate and/or extract collagen and/or gelatin from said animal cell lines and/or tissue explants are needed.

Examples of related art include:

JP2010018575A describes use of jellyfish by a jellyfish extract fraction having cell adhesion inhibitory activity.

JP2004099513A describes a method and a system for extracting and recovering collagen of higher added value through more efficiently treating jellyfish.

JP3696018B2 describes a crude extraction process for useful substances (such as collagen) from jellyfish that includes: crushing jellyfish, shredding the jellyfish into pieces, decomposing the jellyfish, solubilizing the jellyfish, and purifying the jellyfish.

JP2007051191A describes a method for recovering collagen that includes the steps of: freezing jellyfish, thawing the frozen jellyfish to activate an endogenous enzyme of jellyfish to start the decomposition reaction of jellyfish, mixing the thawed jellyfish to solubilize the collagen of jellyfish in a native state to form a neutral salt solution containing native collagen, and recovering the native collagen from the neutral salt solution.

JP2008031106A describes a method that comprises a low-temperature storage step for storing jellyfish at a low temperature for activating an endogenous enzyme of jellyfish to cause it to initiate a decomposition reaction of the jellyfish and solubilizing collagen of the jellyfish in an unmodified state to form a neutral salt solution containing unmodified collagen. The method also includes a recovery step for recovering the unmodified collagen from the neutral salt solution.

WO2014157854A1 and U.S. Published Patent Application No. 2016/0052962 A1 describe a method for isolating collagen from jellyfish through use of radiation.

WO2015005830A1 describes a method for producing collagen from jellyfish.

WO2015012682A2 describes an improved process for extracting collagen from aquatic animals (such as jellyfish), comprising alkaline treatment, followed by acidic treatment in combination with an orderly sequence of physical and/or mechanical treatments and precipitation of collagen using a salt solution. The process increases the yield and quality of the collagen while decreasing the production time and is more cost-effective than the processes known heretofore.

WO2018220396A1 describes hydrolyzed collagen types I, II, and V powder compositions, method of preparing the compositions, and use of the compositions in treating a variety of ailments. The collagen is derived from an organism, such as: jellyfish, anemone, echinoderms, limpets, mussels, sea cucumbers, bovine, porcine, rodent, equine or finfish. The jellyfish may be selected from the list consisting of Rhizostomas pulmo, Rhopilema esculentum, Rhopilema nomadica, Stomolophus meleagris, Aurelia sp., Nemopilema nomurai or a combination thereof.

Some systems exist to extract collagen and/or gelatin from an animal, such as a jellyfish. However, such systems harm the animal. Moreover, the means of operation of such systems are substantially different from the present disclosure, as the other inventions fail to solve all the problems taught by the present disclosure.

### SUMMARY OF THE EMBODIMENTS

The present invention and its embodiments relate to methods to produce collagen and/or gelatin in animal tissue and/or cell cultures and isolate and/or extract collagen and/or gelatin from said animal tissue and/or cell cultures. In particular, the present invention relates to methods to produce collagen and/or gelatin in marine animal tissue and/or cell cultures and isolate and/or extract collagen and/or gelatin from said marine animal tissue and/or cell cultures.

A first embodiment of the present invention describes a method. The method includes numerous process steps, such as: establishing and/or culturing a (continuous) cell line of an animal. The animal may be an invertebrate animal or a vertebrate animal. In other examples, the animal may be a marine animal, a porcine animal, a bovine animal, or an avian animal, among other examples not explicitly listed herein. In some examples where the animal is a marine animal, the marine animal may be: a jellyfish, an anemone, an echinoderm, a limpet, a mussel, a marine sponge, or a sea cucumber, among other examples not explicitly listed herein. The jellyfish may be Rhizostomas pulmo, Rhopilema esculentum, Rhopilema nomadica, Stomolophus meleagris, Aurelia sp., or Nemopilema nomurai.

The method may also include engineering and/or genetically modifying the (continuous) cell line of the animal and isolating a first collagen and/or a first gelatin from the (continuous) cell line of the animal. The first collagen may be endogenous collagen or exogenous collagen. Optionally, the method may include modifying and/or genetically engineering DNA sequences of the first collagen for an application, such as: a food application, a beverage application, a cosmetic application, a medicinal application, a healthcare application, and/or a pharmaceutical application, among other applications not explicitly listed herein.

In some examples, a second collagen and/or a second gelatin is replaced in a product with the first collagen and/or the first gelatin. In other examples, the method further includes modifying and/or genetically engineering DNA sequences of the first collagen to increase a production of the first collagen in cells of the animal. Such modification and/or genetic engineering occurs using Clustered Regularly-Interspaced Short Palindromic Repeats (CRISPR) technology.

A second embodiment of the present invention describes a method. The method includes numerous process steps, such as: utilizing a media to cultivate a (continuous) cell line of an animal. The animal may be an invertebrate animal or a vertebrate animal. In other examples, the animal may be a marine animal, a porcine animal, a bovine animal, and/or an avian animal, among other examples not explicitly listed herein. The method may also include: extracting collagen from the (continuous) cell line of the animal through use of a material and/or a process. The material may be a buffer, salt, an enzyme, an acid, and/or a base. The enzyme may include collagenase and/or pepsin, among other examples. The process may include freeze-drying and/or lyophilizing.

A third embodiment of the present invention describes a method to extract collagen from a cultured animal explant through use of a material and/or a process. The animal explant includes a vertebrate animal explant and an invertebrate animal explant. In other examples, the animal explant includes an avian animal explant, a bovine animal explant, a porcine animal explant, and/or a marine animal explant, among other examples not explicitly listed herein. The marine animal explant is a jellyfish explant, a jellyfish polyp explant, a jellyfish medusae explant, and/or a marine sponge explant, among other examples not explicitly listed herein.

In general, the present invention succeeds in conferring the following benefits and objectives.

It is an object of the present invention to provide a method to create a (continuous) animal cell line.

It is an object of the present invention to provide a method to create a (continuous) jellyfish cell line.

It is an object of the present invention to provide a method to extract collagen and/or gelatin from the (continuous) animal cell line.

It is an object of the present invention to provide a method to replace traditional collagen and/or gelatin from a bovine or a porcine source with collagen and/or gelatin from the (continuous) animal cell line.

It is an object of the present invention to provide a method to replace traditional collagen and/or gelatin from a bovine or a porcine source with collagen and/or gelatin from the (continuous) jellyfish cell line.

It is an object of the present invention to provide a humane method to extract collagen from the (continuous) animal cell line that does not harm the animal.

It is an object of the present invention to provide a humane method to extract collagen from the (continuous) jellyfish cell line that does not harm the jellyfish.

### BRIEF DESCRIPTION OF THE DRAWINGS

FIG. 1 depicts a cutaway schematic diagram depicting anatomy of a jellyfish, according to at least some embodiments disclosed herein.

FIG. 2 depicts a block diagram of a method, according to at least some embodiments disclosed herein.

FIG. 3 depicts a block diagram of another method, according to at least some embodiments disclosed herein.

FIG. 4 depicts a block diagram of an additional method, according to at least some embodiments disclosed herein.

FIG. 5 depicts images of bovine and porcine cells stained for collagen in a Picsirius red stain in DMEM/F-12, DMEM advanced, and RPMI 1640 advanced media, according to at least some embodiments disclosed herein.

FIG. 6A depicts a graphical representation of collagen production in different media for human cells, according to at least some embodiments disclosed herein.

FIG. 6B depicts a graphical representation of collagen production in different media for bovine cells, according to at least some embodiments disclosed herein.

FIG. 7A depicts a graphical representation of collagen production under different concentrations of vitamin C, with a seeding density of 40,000 bovine cells/well, according to at least some embodiments disclosed herein.

FIG. 7B depicts a graphical representation of collagen production under different concentrations of vitamin C, with a seeding density of 70,000 bovine cells/well, according to at least some embodiments disclosed herein.

FIG. 7C depicts a graphical representation of collagen production under different concentrations of vitamin C, with a seeding density of 40,000 human cells/well, according to at least some embodiments disclosed herein.

FIG. 7D depicts a graphical representation of collagen production under different concentrations of vitamin C, with a seeding density of 70,000 human cells/well, according to at least some embodiments disclosed herein.

FIG. 8 depicts a graphical representation of the effects of ginseng on collagen production in various cell types, according to at least some embodiments disclosed herein.

FIG. 9 depicts a graphical representation of the effects of palmitoleic acid on collagen production in various cell types, according to at least some embodiments disclosed herein.

FIG. 10 depicts a graphical representation of the effects of spirulina on collagen production in various cell types, according to at least some embodiments disclosed herein.

FIG. 11 depicts a graphical representation of the effects of collagen yield based on an addition of one or more factors in commercial bovine and a primary bovine fibroblast cell line of the instant invention, according to at least some embodiments disclosed herein.

FIG. 12 depicts a visual comparison of a primary bovine fibroblast cell line of the instant invention, a commercial bovine fibroblast cell line, a human keloid fibroblast cell line, and a commercial porcine cell line, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Jellyfish Anatomy

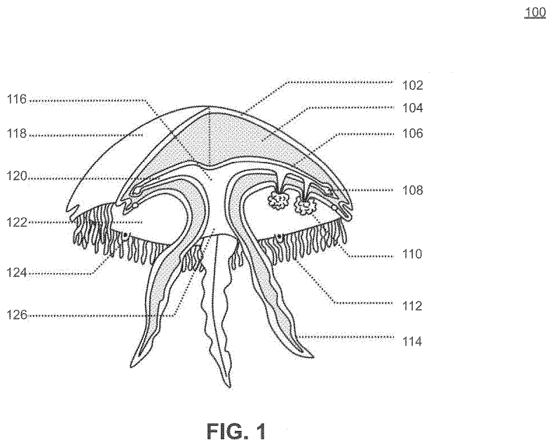

As depicted in FIG. 1, a “jellyfish” 100 is the informal common name given to the medusa-phase of certain gelatinous members of the subphylum Medusozoa, a major part of the phylum Cnidaria. The jellyfish 100 is mainly a free-swimming marine animal with an umbrella-shaped bell and trailing tentacles 112. In general, the jellyfish 100 is composed of three layers: an outer layer (e.g., an epidermis 102), a middle layer (e.g., a mesoglea 104) and an inner layer (e.g., a gastrodermis 106).

The main feature of the jellyfish 100 is the umbrella-shaped bell. The umbrella-shaped bell is a hollow structure consisting of a mass of transparent jelly-like matter (e.g., the mesoglea 104), which forms the hydrostatic skeleton of the animal. The bell can pulsate to provide propulsion for the jellyfish 100. See, Edward E. Ruppert, et al., “Invertebrate Zoology,” 2004, 7th edition, Cengage Learning, Pages 148-174, the entire contents of which are hereby incorporated by reference in their entirety.

Approximately 95% or more of the mesogloea 104 consists of water, but it also contains collagen and other fibrous proteins, as well as wandering amoebocytes which can engulf debris and bacteria. See, Yun-Hwa Hsieh, “Potential of utilizing jellyfish as food in Western countries,” Trends in Food Science & Technology, 2004, 5 (7), Pages 225-229; and Seiya Miura, et al., “Jellyfish Mesogloea Collagen,” The Journal of Biological Chemistry, 1985, Vol. 260, No. 28, Pages 15352-15356, the entire contents of which are hereby incorporated by reference in their entirety. Further, the edge of the bell is often divided into rounded lobes (e.g., lappets), which allow the bell to flex. In the gaps or niches between the lappets are dangling rudimentary sense organs known as rhopalia, and the margin of the bell often bears the tentacles 112. See, Edward E. Ruppert, et al.

Moreover, the jellyfish 100 may also contain a circular canal 108 that runs around the circumference of the bell of the jellyfish 100. A radial canal 120 may radiate away from the stomach and then connect to a ring canal, if present, and then back to the stomach. The radial canal 120, the ring canal (if present), and a stomach or gastric cavity 116 form the gastroendodermal system. Moreover, at the topmost domed surface of the bell of the jellyfish 100 is an exumbrella 118. At the lower surface of the domed surface of the bell of the jellyfish 100 is a subumbrella 122.

Hanging from the center of a subumbrellar 122 is a projection (e.g., a manubrium), that bears a mouth 126 at its terminus. The mouth 126 is often surrounded by four oral arms 114. The stomach is divided into a central chamber and four pouches coming off the sides. The pouches contain gonads 110, or the reproductive organs that produce sperm and/or egg cells. Rhopalia (or rhopalium) 124 are small sensory structures of Scyphozoa (e.g., a typical jellyfish) and Cubozoa (e.g., box jellies).

### Collagen

Collagen is the predominant structural protein in the extracellular matrix of connective tissues in animals and is widely used in tissue regeneration and other industrial applications. See, K. E. Kadler, et al., “Collagens at a Glance,” Journal of Cell Science, 2007, 120, Pages 1955-1958, the entire contents of which are hereby incorporated by reference in their entirety. Collagen may be fibrillar or non-fibrillar. Fibrillar collagen includes Type I, Type II, Type III, Type V, and Type XI. Non-fibrillar collagen includes fibril associated collagens with interrupted triple helices (or FACIT) (e.g., Type IX, Type XII, Type XIV, Type XIX, and Type XXI), short chain collagen (Type VIII and Type X), basement membrane collagen (e.g., Type IV), multiple triple helix domains with interruptions (or Multiplexin) (e.g., Type XV and Type XVIII), membrane associated collagens with interrupted triple helices (or MACIT) (e.g., Type XIII and Type XVII), and others (e.g., Type VI and Type VII). The five most common types of collagen include Type I (e.g., the main component of the organic part of bone), Type II (e.g., the main collagenous component of cartilage), Type III (e.g., the component of reticular fibers), Type IV forms basal lamina, the epithelium-secreted layer of the basement membrane), and Type V (e.g., cell surfaces, hair, and placenta).

Commercially available collagen-based agents are usually derived from bovine and porcine sources. However, collagen of bovine origin are associated with the transmission of bovine spongiform encephalopathy (BSE) (or mad cow disease) and transmissible spongiform encephalopathy (TSE), as well as potential viral vectors that could be transmissible to humans. See, M. Ogawa, et al., “Biochemical Properties of Bone and Scale Collagens Isolated from the Subtropical Fish Black Drum (Pogonia cromis) and Sheepshead Seabream (Archosargus probatocephalus),” Food Chem., 2004, 88(4), Pages 495-501; H. Li, et al., “Studies on Bullfrog Skin Collagen,” Food Chem., 2004, 84(1), Pages 65-9; and J. P. Widdowson J. P., et al., “In Vivo Comparison of Jellyfish and Bovine Collagen Sponges as Prototype Medical Devices,” J. Biomed. Mater. Res. Part B Appl. Biomater, 2018, 106, Pages 1524-1533, the entire contents of which are hereby incorporated by reference in their entirety. Moreover, porcine collagen can also cause religious and/or ethical problems. See, B. Hoyer, et al., “Jellyfish Collagen Scaffolds for Cartilage Tissue Engineering,” Acta Biomater, 2014,10, Pages 883-892, the entire contents of which are hereby incorporated by reference in their entirety. Furthermore, there are growing regulatory concerns around the continued use of mammalian collagens, as they are considered a pathological risk for transmitted diseases, such as avian influenza, swine influenza, and tooth-and-mouth disease. See, F. Subhan, et al., “Marine Collagen: an Emerging Player in Biomedical Applications,” J. Food Sci. Technol., 2015,52, Pages 4703-4707, the entire contents of which are hereby incorporated by reference in their entirety. Some have also shown that different mammalian collagen-based materials induce pro-inflammatory tissue responses due to their purification processes. See, T. Miyata, et al., “Collagen Engineering for Biomaterial Use. Clin. Mater,” 1992,9, Pages 139-148; and J. M. Aamodt, et al., “Extracellular Matrix-Based Biomaterial Scaffolds and the Host Response,” Biomaterials, 2016,86, Pages 68-82, the entire contents of which are hereby incorporated by reference in their entirety.

Due to these deficiencies, marine organisms have gained interest as alternative, non-mammalian collagen sources for biomaterial applications because of potential medical and economic advantages. Interestingly, marine organisms present an attractive alternative, due to lack of BSE risk and potential viral vectors. Specifically, jellyfish have been viewed as one such alternative, since jellyfish are rich in minerals, proteins, and collagen. See, Y. P. Hsieh, et al., “Jellyfish as Food,” Hydrobiologia, 2001,451(1-3), Pages 11-7, the entire contents of which are hereby incorporated by reference in their entirety.

The isolation and characterization of collagen from different fish and jellyfish species has been described. See, S. Addad, et al., “Isolation, Characterization and Biological Evaluation of Jellyfish Collagen for Use in Biomedical Applications,” Mar. Drugs, 2011,9, Pages 967-983; Z. Rastian, et al., “Type I Collagen from Jellyfish Catostylus Mosaicus for Biomaterial Applications,” ACS Biomater. Sci. Eng, 2018, 4, Pages 2115-2125; S. Krishnan, et al., “Preparation and Biomedical Characterization of Jellyfish (Chrysaora Quinquecirrha) Collagen from Southeast Coast of India,” Int. J. Pharm. Pharm. Sci, 2013, 5, Pages 698-701; and S. Yamada, et al., “Potency of Fish Collagen as a Scaffold for Regenerative Medicine,” Biomed Res. Int, 2014, Page 302932, the entire contents of which are hereby incorporated by reference in their entirety. Moreover, the usability of marine collagens has already been analyzed and it has been shown that jellyfish collagen is non-toxic and induces a higher cell viability of fibroblasts and osteoblasts compared to bovine collagen. See, S. Addad, et al. Additionally, others have conducted studies that tested different Mediterranean jellyfish species in order to investigate the different methods of collagen purification. See, S. Addad, et al. Based on this study, it was concluded that the best collagen yield was obtained using Rhizostoma pulmo (R. pulmo), and furthermore, upon biological analysis, the cytotoxicity of R. pulmo collagen was no different in comparison to mammalian collagen. See, S. Addad, et al.

Further studies supporting the potential of jellyfish collagen's biocompatibility have been conducted, which included cytotoxicity tests, measurements of pro-inflammatory cytokine secretion and antibody secretion, as well as the population change of immune cells after in vivo implantation. See, S. Addad, et al. In one such study, it was found that the number of dendritic cells (CD11c+) and macrophages (F4/80+) were similar in jellyfish collagen implanted into mice as to those of bovine- and gelatin-implanted mice. See, E. Song, et al., “Collagen Scaffolds Derived from a Marine Source and Their Biocompatibility,” Biomaterials, 2006, 27, Pages 2951-2961, the entire contents of which are hereby incorporated by reference in their entirety. Hence, this study concluded that the jellyfish collagen scaffolds were able to induce a comparable immune response to that caused by bovine collagen or gelatin. See, E. Song, et al.

Additionally, another study reported that the peptides derived from R. esculentum could reduce the blood pressure in spontaneously hypertensive rats and be used as antihypertensive compounds in functional foods. See, X. Liu, et al., “Purification and Characterization of Angiotensin I Converting Enzyme Inhibitory Peptides from Jellyfish Rhopilema esculentum,” Food Res Int., 2013, 50(1), Pages 339-43, the entire contents of which are hereby incorporated by reference in their entirety. Another group reported that proteins isolated from jellyfish R. esculentum showed strong antioxidant activity and might be applied in the food and pharmaceutical industries. See, H. Yu, et al., “In vitro Determination of Antioxidant Activity of Proteins from Jellyfish Rhopilema esculentum,” Food Chem, 2006, 95(1), Pages 123-30, the entire contents of which are hereby incorporated by reference in their entirety.

Jellyfish collagen possesses the common feature of collagen molecules exhibiting a triple helix structure and is resistant to pepsin digestion. See, A. Miki, et al., “Structural and Physical Properties of Collagen Extracted from Moon Jellyfish under Neutral pH Conditions,” Biosci Biotechnol Biochem, 2015, 79, Pages 1603-1607; and B. Hoyer, et al., the entire contents of which are hereby incorporated by reference in their entirety. Moreover, jellyfish collagen, which can be defined as “collagen type 0” due to its homogeneity to the mammalian types I, II, III, V, and IX and its batch-to-batch consistent producibility, is of special interest for different medical applications related to (bone) tissue regeneration as an alternative to mammalian collagen-based biomaterials. See, Iris Flaig, et al., “In Vivo Analysis of the Biocompatability and Immune Responses of Jellyfish Collagen Scaffolds and its Suitability for Bone Regeneration,” International Journal of Molecular Sciences, 2020, 21(12), Page 4518, the entire contents of which are hereby incorporated by reference in their entirety.

Some have shown that collagens extracted from several species of jellyfish exhibit unique functional properties. For example, species of Nemopilema are known to be edible and harmless jellyfish, and their collagen stimulates immune reactions through the TLR4 signaling pathway. See, H. Morishige, et al., “Immunostimulatory Effects of Collagen from Jellyfish in vivo,” Cytotechnology, 2011, 63, Pages 481-492; and A. B. Putra, et al., “Jellyfish Collagen Stimulates Production of TNF-α and IL-6 by J774.1 Cells Through Activation of NF-κB and JNK via TLR4 Signaling Pathway,” Mol Immunol, 2014, 58, Pages 32-37, the entire contents of which are hereby incorporated by reference in their entirety. Others have reported that collagen extracted from this species accelerates cartilage differentiation from mesenchymal stem cells. See, B. Hoyer, et al. Collagen extracted from Aurelia species (moon jellyfish) possesses the unique property of high water solubility that collagens from other species of jellyfish do not. See, A. Miki, et al. Despite these promising benefits, the structures and functions of collagenous proteins in invertebrates, such as jellyfish, have not been fully understood.

Traditional methods to extract collagen from a collagen-containing matter may include numerous process steps. In examples, the phrase “collagen-containing matter” refers to a source material from which the collagen is to be extracted. In some embodiments, the collagen-containing matter is derived from an organism, such as: a jellyfish, an anemone, an echinoderm, a limpet, a mussel, a sea cucumber, a bovine, a porcine, a rodent, an equine, or a finfish. Such method may be described in WO 2018/220396 A1, the contents of which are hereby incorporated by reference in their entirety. One such illustrative method includes: (a) incubating the collagen-containing matter in an acidic solution for at least 1 hour at a temperature in the range of about 4° C. to about 37° C. to form an incubant; (b) diafiltrating the incubant from step (a) to substantially purify solubilized collagen within the incubant, thereby forming a retentate; (c) separating the soluble and insoluble matter of the retentate obtained from step (b) to remove the remaining insoluble matter; and (d) optionally repeating steps (a) and (b) on the remaining insoluble matter, where the soluble matter obtained from step (c) is a substantially pure collagen solution. However such methods harm the animal. As such, humane alternatives are needed to extract collagen from organisms.

### Gelatin

Gelatin is typically derived from denatured collagen via acid hydrolysis, alkaline hydrolysis, and enzyme hydrolysis. Type A and Type B gelatin commonly used in food industry are derived by acid and alkaline processes, respectively. Type A gelatin produced from jellyfish can be used as an alternative source of gelatin for food application. See, U. Rodsuwan, et al., “Functional Properties of Type A Gelatin from Jellyfish (Lobonema smithii),” International Food Research Journal, 2016, 23(2), Pages 507-514, the entire contents of which are hereby incorporated by reference in their entirety.

### Methods

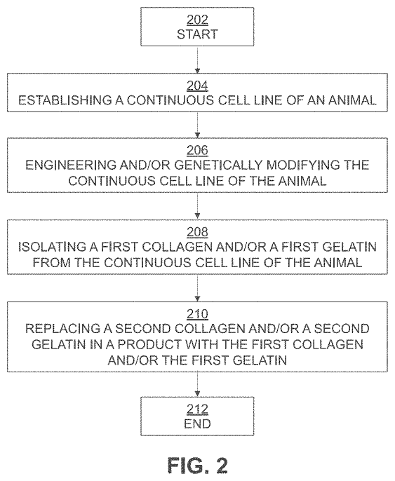

The present invention describes numerous humane methods. A first method is depicted in FIG. 2. The method of FIG. 2 comprises numerous process steps, such as a process step 202 that begins the method. A process step 204 follows a process step 202 that includes establishing and/or culturing a (continuous) cell line of an animal. The animal may be an invertebrate animal or a vertebrate animal. In other examples, the animal may be a marine animal, a porcine animal, a bovine animal, or an avian animal, among other examples not explicitly listed herein. In some examples where the animal is a marine animal, the marine animal may be: a jellyfish, an anemone, an echinoderm, a limpet, a mussel, a marine sponge, or a sea cucumber, among other examples not explicitly listed herein. In other examples, the mussel may be Mytilus edulis and the sea cucumber may be Stichopus mollis. Preferably, the animal may be a jellyfish and the jellyfish may be Rhizostomas pulmo, Rhopilema esculentum, Rhopilema nomadica, Stomolophus meleagris, Aurelia sp., or Nemopilema nomurai.

A process step 206 follows the process step 204 and includes engineering and/or genetically modifying the (continuous) cell line of the animal. The first collagen may be endogenous collagen or exogenous collagen. It should be appreciated that “endogenous collagen” is a natural collagen synthesized by the body of the animal and “exogenous collagen” comes from an external source. Next, a process step 208 follows the process step 206 and includes isolating a first collagen and/or a first gelatin from the (continuous) cell line of the animal. A process step 210 follows the process step 208 and includes replacing a second collagen and/or a second gelatin in a product with the first collagen and/or the first gelatin. A process step 212 follows the process step 210 and concludes the method of FIG. 2.

The method of FIG. 2 may additionally include modifying and/or genetically engineering DNA sequences of the first collagen for an application, such as: a food application, a beverage application, a cosmetic application, a medicinal application, a healthcare application, and/or a pharmaceutical application, among other applications not explicitly listed herein. In other examples, the method further includes modifying and/or genetically engineering DNA sequences of the first collagen to increase a production of the first collagen in cells of the animal. Such modification and/or genetic engineering occurs using Clustered Regularly Interspaced Short Palindromic Repeats (CRISPR) technology.

It should be appreciated that “CRISPR” refers to a family of DNA sequences found in the genomes of prokaryotic organisms, such as bacteria and archaea. These sequences are derived from DNA fragments of bacteriophages that had previously infected the prokaryote and are used to detect and destroy DNA from similar bacteriophages during subsequent infections. As such, these sequences play a key role in the antiviral (i.e. anti-phage) defense system of prokaryotes. See, R. Barrangou, “The roles of CRISPR-Cas Systems in Adaptive Immunity And Beyond,” Current Opinion in Immunology, 2015, 32, Pages 36-41; and Yingxiao Zhang, et al., “The Emerging and Uncultivated Potential of CRISPR Technology in Plant Science,” Natural Plants, 2019, 5, Pages 778-794, the contents of which are hereby incorporated by reference in their entirety. CRISPR technology is a simple yet powerful tool for editing genomes and allows researchers to easily alter DNA sequences and modify gene function. CRISPR technology has numerous potential applications, including correcting genetic defects, treating and preventing the spread of diseases, and improving crops.

It should be appreciated that in some examples, the gelatin may be obtained from jellyfish, such as Aurelia arita. The process for producing gelatin from the jellyfish may include one or more process steps, such as: removing jellyfish parts except the bell, cutting the jellyfish bell into pieces, washing the jellyfish bell with distilled water, soaking the jellyfish bell in hot water to reduce a fat content of the jellyfish bell, cooking the jellyfish bell at about 100° C. for about 30 minutes, soaking the jellyfish bell in an acidic or an alkaline wash (e.g., 20 mM ammonium hydroxide) for about 5 days, straining and boiling the jellyfish bell in distilled water, filtering out any remaining bits of tissue from the jellyfish bell, dehydrating the jellyfish bell, and using a mortar and pestle to grind the jellyfish bell into a powder.

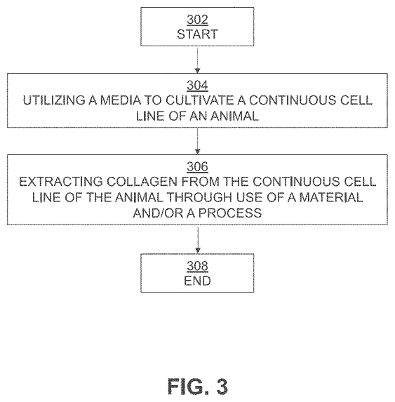

FIG. 3 depicts another method of the present invention. The method of FIG. 3 begins with a process step 302. A process step 304 follows the process step 302 and includes utilizing a medium to cultivate a (continuous) cell line of an animal. The animal may be an invertebrate animal or a vertebrate animal. In other examples, the animal may be a marine animal, a porcine animal, a bovine animal, and/or an avian animal, among other examples not explicitly listed herein. In preferable examples, the animal may be a marine animal, and more specifically, the jellyfish.

A process step 306 follows the process step 304 and includes extracting collagen from the (continuous) cell line of the animal through use of a material and/or a process. The material may be a buffer, salt, an enzyme, an acid, and/or a base. In some examples, the enzyme may include collagenase and/or pepsin, among other examples. The process may include freeze-drying or lyophilizing. As described herein, “lyophilization” or “freeze-drying” is a low temperature dehydration process that involves freezing the product, lowering pressure, then removing the ice by sublimation. See, P. Fellows, “Freeze drying and freeze concentration,” Food processing technology: Principles and practice, 2017, 4th ed., Kent: Woodhead Publishing/Elsevier Science, Pages 929-940, the entire contents of which are hereby incorporated by reference in their entirety. A process step 308 follows the process step 306 and concludes the method of FIG. 3.

In additional examples, the present invention describes a method for extracting collagen from a cultured animal explant through use of a material and/or a process. In some examples, the animal explant includes a vertebrate animal explant and/or an invertebrate animal explant. In other examples, the animal explant includes an avian animal explant, a bovine animal explant, a porcine animal explant, and/or a marine animal explant, among other examples not explicitly listed herein. The marine animal explant is a jellyfish explant, a jellyfish polyp explant, a jellyfish medusae explant, and/or a marine sponge explant, among other examples not explicitly listed herein.

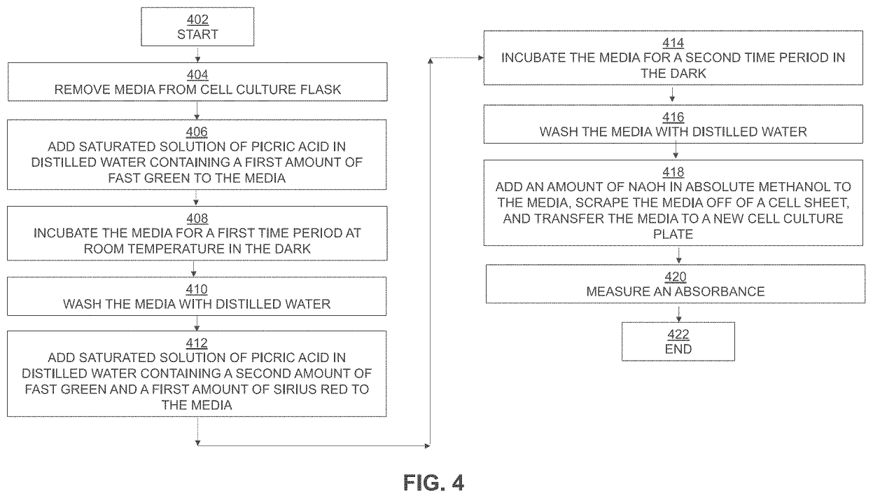

In another example, a collagen detection method using a dual dye assay is described and depicted in FIG. 4. This method measures collagen content of samples using dyes in order to estimate collagen production of adherent cells in cultures without extracting the collagen. Samples may include bovine derma primary fibroblast cell lines or BJ human fibroblast/primary cells, among others not explicitly listed herein.

Materials utilized in the method of FIG. 4 include: about 70% ethanol, sterile water, picric acid (or 2,4,6-trinitrophenol), about 0.1% Fast Green FCF solution in saturated picric acid, about 0.4% Fast Green FCF solution and about 0.11% Sirius Red in the saturated picric acid, and about 0.1% NaOH in methanol. The Fast Green FCF dye is used for total protein and the Sirius Red dye is used for collagen content determination. Equipment utilized with the method of FIG. 4 includes a laminar flow cabinet, a spectrophotometer, and a pipette.

The method of FIG. 4 begins with a process step 402, which is followed by a process step 404. The process step 404 includes removing media from a cell culture flask. The media may include: Minimum Essential Medium (MEM), Dulbecco's Modified Eagle Medium (DMEM)-High glucose, DMEM-F12, FM, DMEM advanced, and/or RPMI 1640 advanced, among others not explicitly listed herein.

A process step 406 follows the process step 404 and includes adding a saturated solution of picric acid in distilled water containing a first amount of the Fast Green dye. In examples, the first amount of the Fast Green dye is about 0.01%. A process step 408 follows the process step 406 and includes incubating the media for a first time period at room temperature in the dark. In examples, the first time period is about 15 minutes. Specifically, this process step may include adding 300 μL/well of 0.01% of the Fast Green dye to twenty-four wells of a test plate and incubating such at room temperature in the dark for about 15 minutes.

A process step 410 follows the process step 408 and includes washing the media with distilled water. At this process step, the washing may occur about 3 to about 4 times. A process step 412 follows the process step 410 and includes adding a saturated solution of picric acid in distilled water containing a second amount of the Fast Green dye and a first amount of the Sirius Red dye to the media. In examples, the second amount of the Fast Green dye is about 0.04% and the first amount of the Sirius Red dye is about 0.11%. More specifically, this process step may include adding 300 μL/well of 0.04% of the Fast Green dye and 0.11% of the Direct Red 80/Sirius Red dye in picric acid.

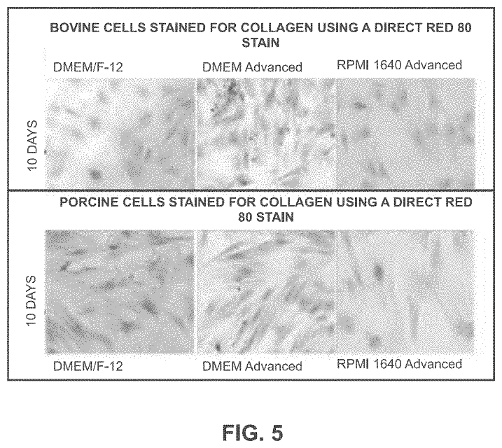

A process step 414 follows the process step 412 and includes incubating the media for a second time period in the dark. The second time period is about 30 minutes. A process step 416 follows the process step 414 and includes washing the media with distilled water about three to about four times. After the process step 416 occurs, the cells may be imaged under a microscope. These images may be depicted in FIG. 5.

A process step 418 follows the process step 416 and includes: adding an amount of NaOH in absolute methanol (1:1) to the media, scraping the media off a cell sheet, and transferring the media to a new cell culture plate. About 0.3 mL of 0.1% NaOH is used in the process step 418.

A process step 420 follows the process step 418 and includes utilizing the spectrophotometer to measure an absorbance at 540 nm and 630 nm. A process step 422 follows the process step 420 to conclude the method of FIG. 4.

It should be appreciated that a green fluorescence probe Col-F may be used. The green fluorescence probe Col-F exhibits an affinity to collagen and elastin, making it a convenient tool for fluorescence three-dimensional imaging of intricate collagenous and elastic structures in fresh and frozen animal tissues. A fluorescence microscope may further be used in this method to analyze the three-dimensional imaging.

It should be appreciated that, based on the method of FIG. 4 and as shown in FIG. 5, primary bovine fibroblasts proliferated faster than bovine cells, primary bovine fibroblasts were found to be a superior source of collagen as compared to primary porcine fibroblasts, and both primary bovine and porcine fibroblasts preferred the DMEM/F-12 media and the DMEM advanced media as compared to the RPMI 1640 advanced media.

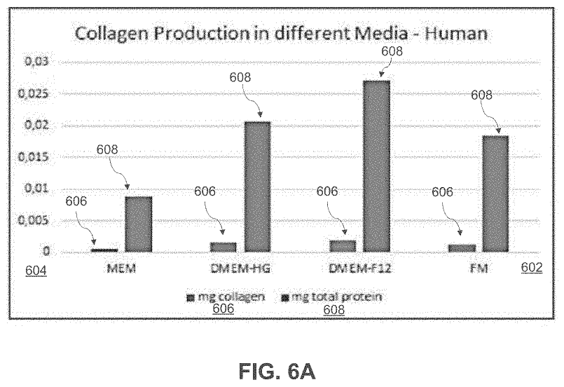

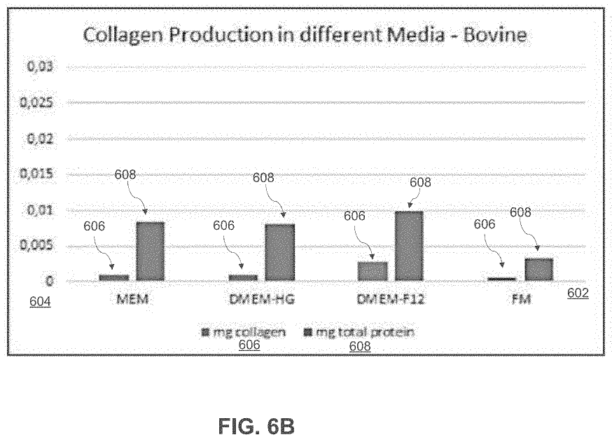

The present invention also provides additional methods to test different media for their effect on collagen production. Specifically, FIG. 6A and FIG. 6B depict collagen production in different media for human and bovine cell lines, respectively. FIG. 6A and FIG. 6B depict an x-axis 602 associated with various media (e.g., MEM, DMEM-High glucose, DMEM-F12 and FM media) and a y-axis 604 associated with collagen production measured in mg. Both the collagen production 606 and the total protein concentration 608 are depicted in FIG. 6A and FIG. 6B. As shown in both FIG. 6A and FIG. 6B, the collagen production 606 and the total protein concentration 608 was highest in the DMEM-F12 media.

The present invention also provides additional methods to test factors for their effect on collagen production within bovine, porcine, human, and other cell lines of interest. Examples of these factors include, but are not limited to: vitamin C, omega-7 fatty acid (or palmitoleic acid), glycine, ginseng extract, zinc sulfate, recombinant human insulin, hydrocortisone hemisuccinate, linoleic acid, asiaticoside, human serum albumin (HSA), lecithin, or spirulina (e.g., an algae supplement), among others. Specifically, researchers have found that cell morphology changes after an addition of vitamin C. See, Richard I. Schwarz, “Collagen I and the Fibroblast: High Protein Expression Requires a New Paradigm of Post-Transcriptional, Feedback Regulation,” Biochemistry and Biophysics Reports, 2015, 3, Pages 38-44, DOI: 10.1016/j.bbrep.2015.07.007, the entire contents of which are hereby incorporated by reference in their entirety. Specifically, Vitamin C is an essential cofactor for the two enzymes required for collagen synthesis: prolyl hydroxylase (e.g., to stabilize the collagen molecule) and lysyl hydroxylase (e.g., to give structural strength cross-linking).

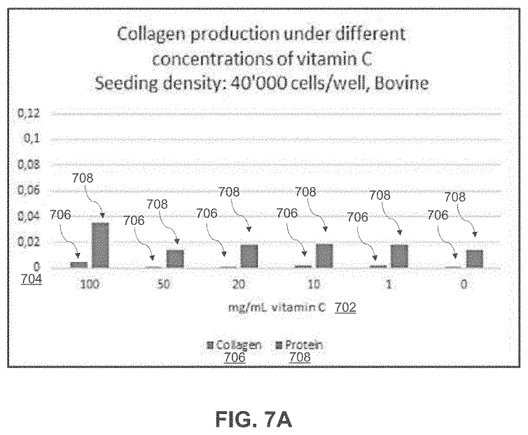

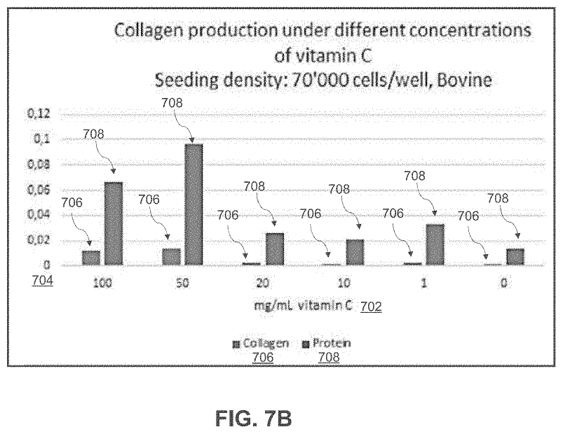

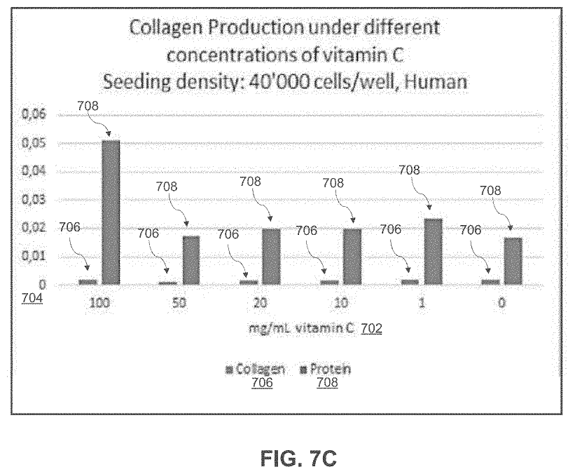

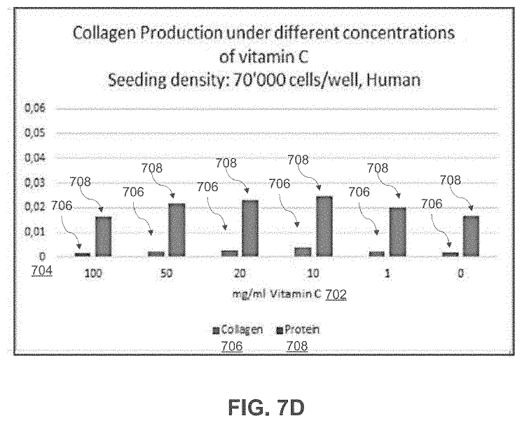

Furthermore, FIG. 7A depicts collagen production under different concentrations of vitamin C, with a seeding density of 40,000 bovine cells/well. FIG. 7B depicts collagen production under different concentrations of vitamin C, with a seeding density of 70,000 bovine cells/well. FIG. 7C depicts collagen production under different concentrations of vitamin C, with a seeding density of 40,000 human cells/well. FIG. 7D depicts collagen production under different concentrations of vitamin C, with a seeding density of 70,000 human cells/well. The experiments associated with the results depicted in FIG. 7A-FIG. 7D were carried out in a 96 well plate.

Specifically, each of FIG. 7A-FIG. 7D include an x-axis 702 associated with a mg/mL concentration of vitamin C and a y-axis 704 that measures a collagen concentration 606 and a protein concentration 608. As shown in FIG. 7A-FIG. 7D, the highest collagen concentration 706 resulted when the concentration of vitamin C was between 10-100 mg/mL.

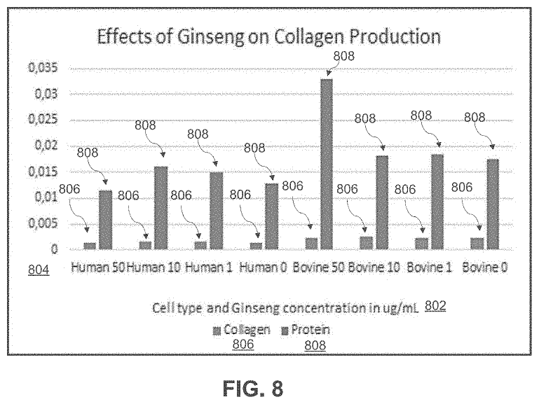

Moreover, FIG. 8 depicts a graph showing effects of ginseng on collagen production in various cell types. FIG. 8 has an x-axis 802 associated with a cell type and a ginseng concentration in μg/mL and a y-axis 704 measuring a collagen concentration 806 and a protein concentration 808.

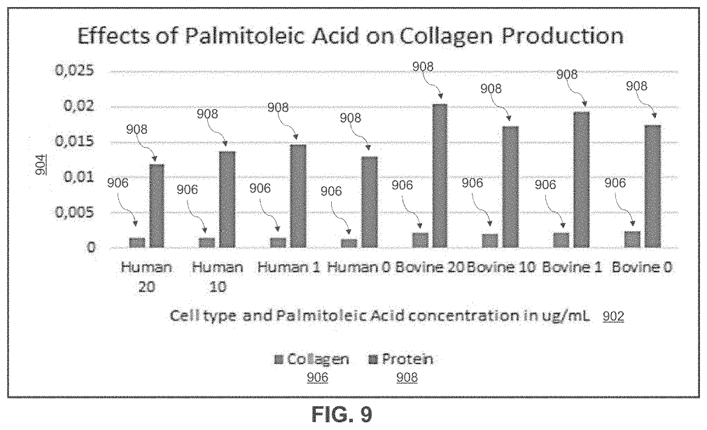

Further, FIG. 9 depicts a graph showing effects of palmitoleic acid on collagen production in various cell types. FIG. 9 has an x-axis 902 associated with a cell type and a palmitoleic acid concentration in μg/mL and a y-axis 904 measuring a collagen concentration 906 and a protein concentration 908.

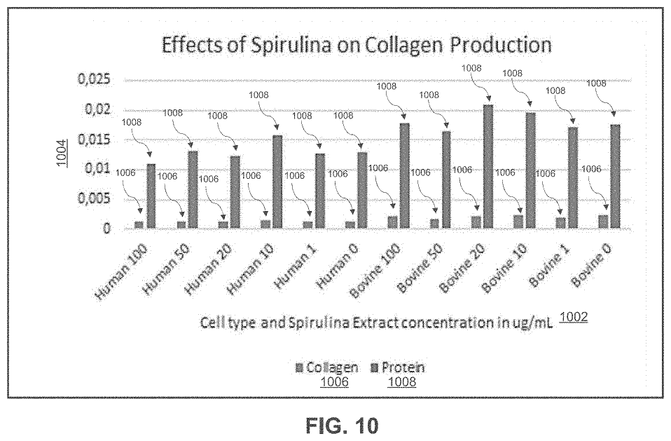

FIG. 10 depicts a graph showing effects of a spirulina extract on collagen production in various cell types. FIG. 10 has an x-axis 1002 associated with a cell type and a spirulina extract concentration in μg/mL and a y-axis 1004 measuring a collagen concentration 1006 and a protein concentration 1008.

As shown in FIG. 8, FIG. 9, and FIG. 10, the ginseng supplement, the palmitoleic acid, and the spirulina extract do not have a large effect on collagen production. However, a total protein content is increased in bovine cells.

In another embodiment, a subset of these factors were evaluated for their ability to enhance collagen production in the commercially available cell lines (bovine, porcine, and human KEL-FIB), as shown in Table 1 below.

TABLE 1

| Factors | Concentration | | | | | | | |
|---|---|---|---|---|---|---|---|---|
| Vitamin C | 0.25 | μM | 0.5 | μM | 1 | μM | 5 | μM |
| Ginseng | 0.07 | ng/ml | 0.125 | ng/ml | 0.25 | ng/ml | 0.5 | ng/ml |
| ZnSO₄ | 0.07 | μM | 0.125 | μM | 0.25 | μM | 0.5 | μM |
| Asiaticoside | 0.07 | μM | 0.125 | μM | 0.25 | μM | 0.5 | μM |
| Glycine | 70 | μM | 125 | μM | 250 | μM | 500 | μM |
| Linoleic Acid | 1 | μM | 20 | μM | 20 | μM | | |

In this embodiment, cells were seeded at approximately 3500-4500 cells per well of a 48 well-plate and were allowed to settle for about 48 hours before adding the factor. After about 48 hours, spent media was removed and replaced with growth media supplemented with the factor of interest. Cells continued to grow for about 3-10 days following initial introduction of the factor and a collagen content was quantified with a Sircol™ Collagen Assay. Additionally, experiments were conducted with and without media/factor replenishment about every 48-72 hours.

Of the factors tested, vitamin C, zinc sulfate, and asiaticoside increased collagen production across the commercially available cell lines (bovine, porcine, and human KEL-FIB). Notably, vitamin C increased collagen production by about 28-36% in the bovine cell lines and by about 50% in the human KEL-FIB cell line.

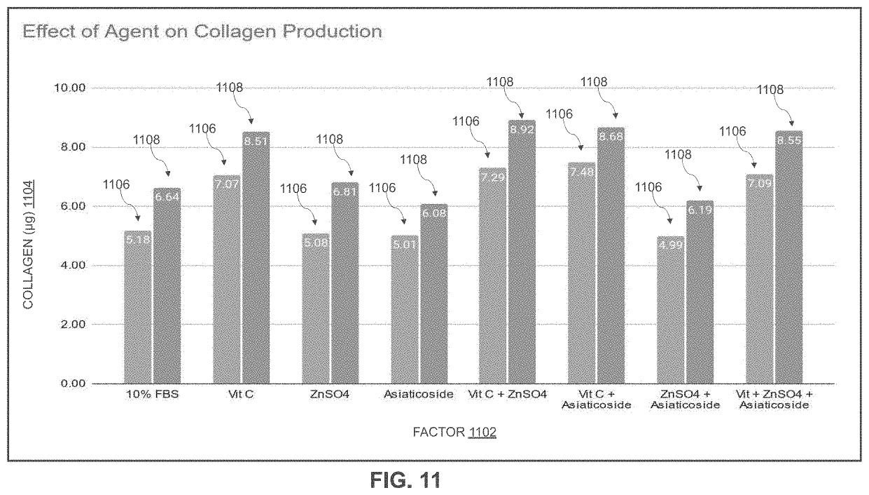

Mixtures of these factors were also tested to evaluate a synergistic effect of the factors on collagen production in the bovine cell lines. FIG. 11 depicts results of this. Specifically, FIG. 11 includes an x-axis 1102 associated with one or more factors and a y-axis associated with collagen concentration in μg. It should be appreciated that a comparison of the collagen yield between a commercial bovine cell line 1106 and a primary bovine fibroblast cell line of the instant invention 1108 for the various factors is depicted after 7 days, where the factors were replenished every 72 hours. As shown in FIG. 11, the combination of vitamin C and asiaticoside was found to increase collagen production to the greatest extent in the commercial bovine cell line 1106 and vitamin C and zinc sulfate was found to increase collagen production the most in the primary bovine fibroblast cell line of the instant invention 1108.

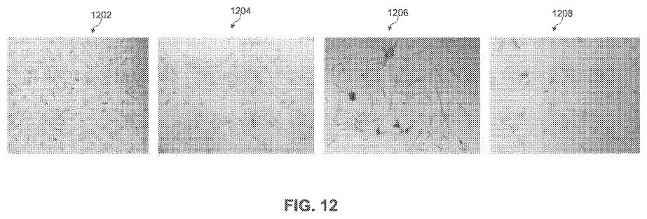

In additional embodiments, methods comparing and contrasting collagen content in various cell lines is also contemplated herein. Specifically, FIG. 12 depicts a visual comparison of a primary bovine fibroblast cell line of the instant invention 1202, a commercial bovine fibroblast cell line 1204, a human keloid fibroblast cell line 1206, and a commercial porcine fibroblast cell line 1208. The cells of FIG. 12 were seeded at approximately the same cell number and incubated for about 72 hours.

The primary bovine fibroblast cell line of the instant invention 1202 outperformed the other commercial cell lines. In particular, the primary bovine fibroblast cell line of the instant invention 1202 outperformed the commercial bovine fibroblast cell line 1204 line by about 17% and about 28% in terms of collagen production after about 3 and about 7 days, respectively. Specifically, the primary bovine fibroblast cell line of the instant invention 1202 is associated with about 7.4 μg collagen, the commercial bovine fibroblast cell line 1204 is associated with about 6.3 μg collagen, the human keloid fibroblast cell line 1206 is associated with about 4.4 μg collagen, and the commercial porcine fibroblast cell line 1208 is associated with about 4.1 μg collagen.

In another embodiment, methods to extract collagen are described herein. In a first method, a method to extract collagen extraction from adherent cells using acetic acid is described. Materials used in this method include: about 70% ethanol, sterile PBS, an about 0.5 M acetic acid solution, and a flask of mostly confluent cells. Equipment used in this method may include: a laminar flow cabinet, a pipette, falcon tubes, a scraper, and Eppendorf tubes.

The first method includes numerous process steps, such as: turning on the flow cabinet about fifteen prior to cell culturing and ensuring that the 0.5 M acetic acid solution is cold and sterile. Next, the method includes moving a cell culture flask or dish from an incubator to the flow cabinet and disposing of a culture medium in a liquid waste container. Then, the method includes washing the cells about three times with 1×PBS. Once washed, the PBS is drained and placed on ice. A small amount of acetic acid is added. The flask is tilted to make sure an entire surface has been in contact with the acetic acid, and then the method includes scraping off an extracellular matrix (ECM) and cells from the bottom of the flask and transferring the solution from the flask into an Eppendorf or falcon tube. The tube is then placed in a 4° C. refrigerator and is incubated for about twenty-four hours. About every 30 minutes, the tube is stirred or rotated. Once the twenty-four hours has passed, the tube is centrifuged at about 15000 rpm for about 30 minutes at +4. The supernatant is collected. The collagen concentration is then measured using an appropriate assay.

In a second example, an extraction method of the ECM of a cell layer using ammonium hydroxide is described. This method lyses and removes cells from a culture flask or dish in order to extract the ECM. Materials used in this method include: about 70% ethanol, sterile PBS, sterile deionized water, a stock solution of ammonium hydroxide, and a flask of mostly confluent cells. Equipment used in this method includes: a laminar flow cabinet, a pipette, falcon tubes, and a scraper.

This method includes numerous process steps, such as: turning on the flow cabinet at least 15 minutes prior to cell culturing. Next, the method includes preparing a 20 mM solution of ammonium hydroxide. Ammonium hydroxide at 1M is usually stored at 4° C. Then, about 300 μL of ammonium hydroxide is added to 14.7 mL sterile water and mixed to prepare 15 mL of 20 mM NH₄OH. Then, the method includes moving the cell culture flask or dish from the incubator to the flow cabinet and disposing of the culture medium in the liquid waste container.

Next, the method includes washing the cells about three times with PBS. Once washed, the PBS is drained and an appropriate amount of ammonium hydroxide is added. The flask is then incubated at room temperature for about 5 minutes. Every minute, the flask is shook gently to make sure all cells get lysed.

Next, the ammonium hydroxide and lysed cells are disposed, leaving only the adhered ECM in place. Now, the ECM is free of cells and can be extracted using scrapers and stored at 4° C. in sterile water, or new cells can be plated on the ECM layer.

Moreover, the present invention describes numerous methods to characterize the purified collagen, such as physicochemical characterization, texture analysis, bloom strength, etc. and other methods known to those having ordinary skill in the art. As an example, Blue Native PAGE and Western Blotting may be used to characterize the collagen isolated from cultured cells.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method comprising:
establishing a (continuous) cell line of an animal;
engineering and/or genetically modifying the (continuous) cell line of the animal;
isolating a first collagen and/or a first gelatin from the (continuous) cell line of the animal; and
replacing a second collagen and/or a second gelatin in a product with the first collagen and/or the first gelatin.

2. The method of claim 1, wherein the animal is selected from the group consisting of: an invertebrate animal and a vertebrate animal.

3. The method of claim 1, wherein the animal is selected from the group consisting of: a marine animal, a porcine animal, a bovine animal, and an avian animal.

4. The method of claim 3,
wherein the animal is a marine animal, and
wherein the marine animal is selected from the group consisting of: a jellyfish, an anemone, an echinoderm, a limpet, a mussel, a marine sponge, and a sea cucumber.

5. The method of claim 4, wherein the jellyfish is selected from the group consisting of: Rhizostomas pulmo, Rhopilema esculentum, Rhopilema nomadica, Stomolophus meleagris, Aurelia sp., Nemopilema nomurai, and a combination thereof.

6. The method of claim 1, wherein the first collagen is endogenous collagen or exogenous collagen.

7. The method of claim 1, wherein the first collagen is human collagen.

8. The method of claim 6, wherein the exogenous collagen is produced in animal cells.

9. The method of claim 8,
wherein the animal cells are selected from the group consisting of: avian cells, porcine cells, bovine cells, and marine cells,
wherein the marine cells are selected from the group consisting of: marine invertebrate cells and marine vertebrate cells,
wherein the marine invertebrate cells are jellyfish cells, and
wherein the marine vertebrate cells are fish cells.

10. The method of claim 1, further comprising:
modifying and/or genetically engineering DNA sequences of the first collagen for an application.

11. The method of claim 10, wherein the application is selected from the group consisting of: a food application, a beverage application, a cosmetic application, a medicinal application, a healthcare application, and a pharmaceutical application.

12. The method of claim 1, further comprising:
modifying and/or genetically engineering DNA sequences of the first collagen to increase a production of the first collagen in cells of the animal.

13. The method of claim 12, wherein the modification and/or the genetic engineering of the DNA sequences of the first collagen to increase the production of the first collagen in the cells of the animal occurs using Clustered Regularly Interspaced Short Palindromic Repeats (CRISPR) technology.

14. A method comprising:
utilizing a media to cultivate a (continuous) cell line of an animal; and
extracting collagen from the (continuous) cell line of the animal through use of a material and/or a process.

15. The method of claim 14, wherein the animal is selected from the group consisting of: an invertebrate animal and a vertebrate animal.

16. The method of claim 14, wherein the animal is selected from the group consisting of: a marine animal, a porcine animal, a bovine animal, and an avian animal.

17. The method of claim 14, wherein the material is selected from the group consisting of: a buffer, an enzyme, an acid, and a base.

18. The method of claim 17, wherein the enzyme is selected from the group consisting of: collagenase and pepsin.

19. The method of claim 14, wherein the process comprises lyophilizing.

20. A method to extract collagen from a cultured animal explant through use of a material and/or a process.

21. The method of claim 20, wherein the animal explant is selected from the group consisting of: a vertebrate animal explant and an invertebrate animal explant.

22. The method of claim 20, wherein the animal explant is selected from the group consisting of: an avian animal explant, a bovine animal explant, a porcine animal explant, and a marine animal explant.

23. The method of claim 22, wherein the marine animal explant is selected from the group consisting of: a jellyfish explant, a jellyfish polyp explant, a jellyfish medusae explant, and a marine sponge explant.

24. A collagen detection method comprising:
removing media from a cell culture flask;
adding a saturated solution of picric acid in distilled water containing a first amount of a first dye;
incubating the media for a first time period at room temperature in the dark;
washing the media with distilled water;
adding a saturated solution of picric acid in distilled water containing a second amount of the first dye and a first amount of a second dye to the media;
incubating the media for a second time period at room temperature in the dark;
washing the media with the distilled water;
adding an amount of sodium hydroxide in absolute methanol to the media;
transferring the media to a new cell culture plate; and
utilizing a spectrophotometer to measure an absorbance of the collagen at various wavelengths.

25. The collagen detection method of claim 24, wherein the media is selected from the group consisting of: Minimum Essential Medium (MEM), Dulbecco's Modified Eagle Medium (DMEM)-High glucose, DMEM-F12, FM, DMEM advanced, and RPMI 1640 advanced.

26. The collagen detection method of claim 24, wherein the first dye is a Fast Green dye, and wherein the second dye is a Sirius Red dye.

27. A method to test an effect of a factor on collagen production in a cell line, the method comprising:
seeding cells of a cell line in a well-plate;
after a first time period, replacing spent media with growth media supplemented with a factor; and
quantifying a collagen content using an assay to determine an effect of the factor on collagen production in the cell line.

28. The method of claim 27, wherein the factor is selected from the group consisting of: vitamin C, omega-7 fatty acid, glycine, ginseng extract, zinc sulfate, recombinant human insulin, hydrocortisone hemisuccinate, linoleic acid, asiaticoside, human serum albumin (HSA), lecithin, and spirulina.

29. The method of claim 27, wherein the cell line is selected from the group consisting of: a bovine cell line, a porcine cell line, and a human cell line.
